(19) United States
(12) Patent Application Publication
(10) Pub. No.: US 2022/0287928 A1
(43) Pub. Date: Sep. 15, 2022

(54) ALGINATE-BASED FACIAL MASK

(71) Applicant: BEIERSDORF AG, Hamburg (DE)

(72) Inventors: Franziska BURR, Hamburg (DE); Thomas RASCHKE, Pinneberg (DE); Heike FOELSTER, Hamburg (DE); Stefan GALLINAT, Wedel (DE); Petra SCHOENDIENST, Tornesch (DE); Svenja LUEGGER, Hamburg (DE)

(21) Appl. No.: 17/629,854

(22) PCT Filed: Jul. 27, 2020
(86) PCT No.: PCT/EP2020/071078

§ 371 Date: May 26, 2022

(30) Foreign Application Priority Data
Jul. 26, 2019 (DE) 10 2019 211 138.6
Oct. 28, 2019 (DE) 10 2019 007 508.0

(51) Int. Cl. A61K 8/02; A61K 8/23; A61K 8/24; A61K 8/34; A61K 8/35; A61K 8/37; A61K 8/73; A61K 8/891; A61K 8/92; A61K 8/99; A61Q 19/00

(52) U.S. Cl. CPC A61K 8/0212; A61K 8/23; A61K 8/24; A61K 8/345; A61K 8/35; A61K 8/375; A61K 8/733; A61K 8/891; A61K 8/922; A61K 8/99; A61Q 19/00; A61K 2800/31

(57) ABSTRACT

The invention relates to an alginate-based facial mask, provided in the form of a water-free cosmetic composition that comprises a) at least one water-soluble alginate, b) at least one calcium salt, c) at least one oil, d) at least one emulsifier selected from the group polyglyceryl-(4) isostearate, polyglyceryl-(10) octanoate/caprate, polyglyceryl-(10) stearate, PEG-7 glyceryl cocoate, polyglyceryl-(3) methyl glucose distearate and polyglyceryl-(4) laurate.

Cosmetic products generally serve not only to look beautiful and attractive, but with their effect they contribute decisively to an increased self-esteem and well-being of people. Accordingly, a wide variety of cosmetic products are used for daily cleaning and care of human skin.

A popular form of facial skin care involves the application of masks, which can either refresh, cleanse or even regenerate the skin.

In the prior art, various technologies for masks are known, between which the skilled person distinguishes.

Known are, for example, sheet masks, where the product consists of thin fleece, which is soaked with an active ingredient solution. The sheets are individually packaged and unfolded for use before being applied to the facial skin. The advantage of these products is that the use of the fleece means that the active ingredient solution cannot be smeared, or only to a limited extent. Furthermore, the provision of recesses in the nonwoven fabric means that the eyes, nostril and mouth can remain uncovered.

Furthermore, cleansing masks based on clay or carbon particles are known, which are applied to the facial skin as a viscous or paste-like preparation. These cleansing masks are often also referred to as clay masks. When applied, the ingredients stimulate the blood circulation of the facial skin and excess sebum is absorbed.

Active ingredient masks are also known which are applied to the skin as a viscous or paste-like preparation and are removed with a cloth after use. The preparation retains its viscous or pasty properties. These masks are often based on water-containing emulsions. The advantage of these active ingredient masks is that active ingredients, such as Q10, tea extracts, hyaluronic acid and plant extracts, can also be incorporated in higher concentrations. A disadvantage is the fact that the preparations smear and thus eye contact is possible or clothing is contaminated.

Another category of known masks are the so-called peel-off masks based on alginates. Such masks are mostly based on an anhydrous powder comprising an alginate and a calcium salt. Alginate is a polysaccharide consisting of the two uronic acids α-L-guluronic acid (GulUA) and β-D-mannuronic acid (ManUA), which are 1,4-glycosidically linked in alternating ratios to form linear chains. It forms homopolymeric regions in which mannuronic acid or guluronic acid are present as blocks. These blocks are called GG or MM blocks. To apply the mask, water is added to the anhydrous powder so that the alginate and calcium salt are dissolved. Gelation then takes place in aqueous solution. This gelation occurs due to the incorporation of calcium ions into the zigzag structure of the GG blocks. The zigzag structure of another alginate molecule is then deposited on this zone. This leads to the formation of three-dimensional structures. Because this reaction with the calcium occurs very abruptly, complexing agents are added to the anhydrous powder to delay curing or gelation, so that the consumer has sufficient time to apply the mask before gelation. After gelation and application to the skin, the consumer can peel off the mask as a film.

Known products of the latter category are sold, among others, by the company Sephora under the trade name Sephora Collection Rubber Mask. The powder is filled with water and the resulting preparation is applied to the skin. Furthermore, EP 0 045 493 A2 describes such products. A disadvantage, however, is the fact that these powder preparations do not contain any skin-caring components, such as glycerin or nourishing oils.

This problem can be solved by offering the customer a cosmetic product comprising a first anhydrous preparation containing at least an alginate and/or algin, a calcium salt, oil components and an emulsifier, and a second hydrous preparation which may be present, inter alia, as an emulsion. However, both preparations must be packaged separately and must not be brought into contact until application, to initiate gelation. An example of such a product is known under Mintel GNPD registration number 5855581. An advantage of this product is that water-soluble and water-insoluble care substances may be included. A disadvantage, however, is the fact that more packaging material has to be used, since both preparations have to be packaged separately. This is not acceptable from an environmental point of view.

Consequently, it remains desirable to provide a cosmetic product for use as an alginate-based mask which can be provided to the customer in a single packaging material and which simultaneously comprises water-soluble and water-insoluble skin care substances. Furthermore, it is desirable to reduce the weight of the cosmetic product until it is used by the consumer so that as little CO₂ as possible is consumed during transportation after manufacture. Furthermore, the cosmetic product should enable a homogeneous paste to be obtained for application to the skin that does not exhibit instabilities due to phase separation.

Surprisingly, it has now been found that these requirements can be solved by the present invention.

The invention relates to an anhydrous cosmetic composition comprising:
a) at least one water-soluble alginate,
b) at least one calcium salt,
c) at least one oil,
d) at least one emulsifier selected from the group polyglyceryl-4 isostearate, polyglyceryl-10 caprylate/caprate, polyglyceryl-10 stearate, PEG-7 glyceryl cocoate, polyglyceryl-3 methyl glucose distearate and polyglyceryl-4 laurate.

The invention also relates to a method for preparing a cosmetic preparation for use as a mask which can be peeled off after application to the skin, comprising the steps of:
providing the anhydrous cosmetic composition according to the invention,
mixing the composition with water to provide an emulsion for application to the skin.

For the purposes of the present invention, the term “anhydrous” means that no added water is contained, whereas water of crystallization may be present. Water of crystallization or water of hydration is the term for water which occurs bound in the crystalline solid.

The composition according to the invention is surprisingly particularly easy to mix with water, advantageously by shaking, to form an emulsion that can be applied to the skin to form the mask. Since water can be added by the consumer at will from the home tap, only one packaging material is needed to create an alginate-based mask.

It was also surprising that skin-moisturizing active ingredients, such as polyols, in particular, could also be incorporated into the composition without causing instability when mixed with water. Consequently, it was possible to provide a composition that contains both polyols and lipophilic active ingredients and can be supplied in a single packaging material.

Advantageously, the composition of the invention therefore comprises at least one polyol. It is preferred if the polyol is selected from the group propylene glycol, butylene glycol, glycerin and sorbitol, wherein the use of glycerin is particularly preferred.

The total proportion of polyols is advantageously from 15 to 50 wt %, preferably from to 20 to 45 wt %, and particularly preferably from to 31 to 42 wt %, based on the total weight of the composition.

It is particularly advantageous if glycerin is included as a polyol and the total proportion of glycerin is from to 15 to 50 wt %, preferably from to 20 to 45 wt %, and more preferably from 31 to 42 wt %, based on the total weight of the composition.

According to the invention, the composition comprises at least one alginate, which is advantageously selected from alginic acid and/or salts thereof. It is particularly advantageous if sodium alginate is included.

Advantageously, the total proportion of the at least one water-soluble alginate is from 5 to 30 wt %, preferably from 10 to 25 wt %, and more preferably from 12 to 18 wt %, based on the total weight of the composition.

It is particularly advantageous if sodium alginate is included as the water-soluble alginate and the total proportion of sodium alginate is from 5 to 30 wt %, preferably from 10 to 25 wt %, and more preferably from 12 to 18 wt %, based on the total weight of the composition.

Furthermore, the composition comprises at least one calcium salt. In this context, the total proportion of calcium salts is preferably from 5 to 20 wt %, preferably from 8 to 17 wt %, and in particular preferably from 10 to 15 wt %, based on the total weight of the composition.

It is particularly advantageous if calcium sulphate is included as the calcium salt. Consequently, it is also particularly advantageous if calcium sulfate is present as a water-soluble calcium salt and the total proportion of calcium sulfate is from 5 to 20 wt %, preferably from 8 to 17 wt %, and more preferably from 10 to 15 wt %, based on the total weight of the composition.

It is also advantageous if calcium sulfate dihydrate is included as the calcium salt. Consequently, it is also particularly advantageous if calcium sulfate dihydrate is included as a water-soluble calcium salt and the total proportion of calcium sulfate dihydrate is from 5 to 20 wt %, preferably from 8 to 17 wt %, and particularly preferably from 10 to 15 wt %, based on the total weight of the composition.

Accordingly, it is advantageous if calcium sulfate and/or calcium sulfate dihydrate is included as the calcium salt, the total proportion of these calcium salts preferably being from 5 to 20 wt %, preferably from 8 to 17 wt %, and in particular preferably from 10 to 15 wt %, based on the total weight of the composition.

The composition according to the invention further comprises at least one oil. According to the invention, substances referred to as emulsifiers or surfactants are not regarded as oils.

Emulsifiers are all substances listed in the International Cosmetic Ingredient Dictionary and Handbook, Thirteenth Edition 2010, (ISBN 1-882621-47-6) under the designation “emulsifying agent”. Surfactants are all substances which are listed in the International Cosmetic Ingredient Dictionary and Handbook, Thirteenth Edition 2010, (ISBN 1-882621-47-6) under the designation “surfactant”.

Advantageously, the proportion of the at least one oil is from 2 to 30 wt %, preferably from 6 to 25 wt %, and more preferably from 12 to 22 wt %, based on the total weight of the composition.

The oils can be advantageously chosen from the natural or synthetic oils.

Beneficial natural oils are selected from the group of sunflower oil (Helianthus annuus seed oil), rape oil (canola Oil), soybean oil (glycine soya oil), olive oil (Olea europaea fruit oil), almond oil (Prunus amygdalus dulcis oil), avocado oil (Persea gratissima oil), walnut oil (Junglans regia seed Oil), peach kernel oil (Prunus persica kernel oil), apricot kernel oil (Prunus armeniaca kernel oil), sesame oil (Sesamum indicum seed oil), camellia oil (Camelia oleifera/Camelia sasanqua), evening primrose Oil (Oenothera biennis), macadamia nut oil (Macadamia intergrifolia seed oil), thistle oil (Silybum marianum seed oil), wheat germ oil (Triticum vulgare germ oil), palm kernel oil (Elaeis guineensis kernel oil), palm oil (Elaeis guineensis oil), grape seed oil (Vitis vinifera seed oil), argan oil (Argania spinosa seed oil), peanut oil (Arachis hypogaea oil), pumpkin seed oil (Cucurbita pepo seed oil), ricinus oil (Ricinus communis seed oil), Jojoba oil, rice germ oil (Oryza sativa bran oil), vegetable oil (Olus oil) and/or coconut oil. Jojoba oil is particularly preferred.

Advantageous synthetic oils are selected from isopropyl palmitate, isopropyl stearate, iso-decyl neopentanoate, cetearyl isonanoate, C12-15 alkyl benzoate, ethyl hexyl stearate, caprylic capric triglyceride, cocoglycerides, octyl cocoate, dibutyl adipate, dicaprylyl ether, octyl dodecanol, butylene glycol dicaprylate/dicaprate, dicaprylyl carbonate, octyl palmitate

Furthermore, in one embodiment it is advantageous if at least dimethicone is contained as oil. Other advantageous embodiments are characterized in that no silicone oils are contained.

It is particularly advantageous if caprylic/capric triglyceride is included as the oil.

Furthermore, ester oils selected from the group consisting of ethyl hexyl stearate, octyl cocoate, cocoglycerides, dicaprylyl carbonate, cetearyl isononanoate, C12-15 alkyl benzoate and isopropyl palmitate are advantageously used as oils.

If at least one oil selected from the group ethyl hexyl stearate, octyl cocoate, cocoglycerides, dicaprylyl carbonate, caprylic capric triglyceride, cetearyl isononanoate, C12-15 alkyl benzoate and isopropyl palmitate is present, it is advantageous according to the invention if the total proportion of these oils is from 5 to 25 wt %, preferably from 6.5 to 24.5 wt % and more preferably from 12.5 to 21.5 wt %, based on the total weight of the composition.

Where dicaprylyl carbonate is present, the proportion of dicaprylyl carbonate is preferably from 6.5 to 24.5 wt %, and more preferably from 12.5 to 21.5 wt %, based on the total weight of the composition.

Furthermore, the composition contains at least one emulsifier selected from the group poly-glyceryl-4 isostearate, polyglyceryl-10 caprylate/caprate, polyglyceryl-10 stearate, PEG-7 glyceryl cocoate, polyglyceryl-3 methyl glucose distearate and polyglyceryl-4 laurate. Poly-glyceryl-4 isostearate is particularly preferred.

Advantageously, the total proportion of the at least one emulsifier selected from polyglyceryl-4 isostearate, polyglyceryl-10 caprylate/caprate, polyglyceryl-10 stearate, PEG-7 glyceryl cocoate, polyglyceryl-3 methyl glucose distearate and polyglyceryl-4 laurate is from 0.5 to 11 wt %, preferably from 1 to 6.5 wt %, and in particular preferably from 2 to 4.5 wt %, in each case based on the total weight of the composition.

It is also advantageous if a complexing agent for calcium ions is additionally included. Advantageously, this complexing agent is selected from the group of pyrophosphates such as di-, tri- or tetrasodium pyrophosphates, as well as EDTA, polylactic acid, iminodisuccinate and sodium citrate. Tetrasodium pyrophosphate is particularly preferred.

The proportion of complexing agents for calcium ions, in particular the proportion of tetrasodium pyrophosphate, is advantageously from 0.2 to 5 wt %, preferably from 0.8 to 4 wt % and more preferably from 1.5 to 3.5 wt %, based on the total weight of the composition.

Furthermore, it is advantageous if the composition according to the invention contains silica, wherein the proportion of silica is advantageously in the range from 1 to 20 wt %, preferably 5 to 16.5 wt %, and in particular preferably from 7.5 to 14.5 wt %, based on the total weight of the composition.

Furthermore, it is advantageous if the composition according to the invention contains diatomaceous earth, wherein the proportion of diatomaceous earth advantageously is in the range from 1 to 20 wt %, preferably from 5 to 16.5 wt %, and more preferably from 7.5 to 14.5 wt %, based on the total weight of the composition.

Furthermore, it is advantageous if the composition contains further active ingredients selected from the group glycyrrhetinic acid, arctiin, folic acid, coenzyme Q10 (ubiquinone), alpha-glucosylrutin, carnitine, carnosine, caffeine, natural and/or synthetic isoflavonoids, glyceryl-glucose, creatine, creatinine, taurine, tocopherol, tocopherol acetate, vitamin C, vitamin C phosphate, vitamin C palmitate, niacinamide, vitamin A palmitate, retinol, panthenol, Glycyrrhiza inflata root extract, licochalcone A, 4-butylresorcinol, N-[(2,4-dihydroxyphenyl)thiazol-2-yl] isobutyramide, honociol and magnolol (also as a component of magnolia extracts), hyaluronic acid and/or silymarin (milk thistle extract).

The composition according to the invention may further contain colorants and/or color pigments. The colorants and color pigments can be selected from the corresponding positive list of the Cosmetics Directive or the EC list of cosmetic colorants. In most cases, they are identical to the colorants approved for foodstuffs. Advantageous color pigments are for example titanium dioxide, mica, iron oxides (e.g. Fe2O3, Fe3O4, FeO (OH)) and/or tin oxide. Advantageous colorants are, for example, carmine, Berlin blue, chromium oxide green, ultramarine blue and/or manganese violet. It is particularly advantageous to select the colorants and/or color pigments from the following list. The color index numbers (CIN) are taken from the Rowe Colour Index, 3rd edition, Society of Dyers and Colourists, Bradford, England, 1971.

Furthermore, it is advantageous if, for use in the process according to the invention, the composition according to the invention is mixed with water in a weight ratio to water of from 10:90 to 60:40, preferably from 15:85 to 40:60, and in particular preferably from 20:80 to 35:65. During mixing, a homogeneous composition is obtained.

The mask composition obtained in this way is advantageously applied to the skin within 10 minutes after mixing. After application, the composition is removed from the skin again as a solid mass.

It has been shown here that the mask composition obtained according to the invention is surprisingly well suited for taking negative prints of the skin, so that the skin structure can be analyzed and documented.

In a further preferred embodiment, the mask composition according to the invention comprises at least one probiotically active bacterial strain which, when colonized on the skin, has a positive effect on skin health. As used herein, “probiotically active” means that the bacterial strain in question imparts health-promoting effects when administered to humans in sufficient quantities. In the present case, the term is used in connection with those bacterial strains which, after administration to the skin, colonize on the skin, i.e. which multiply by cell division, and positively influence skin health by their presence in the skin flora.

In particular, bacteria of the genus Staphylococcus and Cutibacterium are considered as probiotically active bacterial strains in the context of the present invention. These genera are those which are regularly represented in a normal human skin flora. In a particularly preferred embodiment, the at least one probiotically active bacterial strain belongs to a species selected from the group consisting of Staphylococcus epidermidis, Staphylococcus hominis and Cutibacterium acnes.

Staphylococcus epidermidis and Staphylococcus hominis are coagulase-negative, facultative pathogenic gram-positive bacteria that colonize the skin and mucous membranes of humans. Preferably, the at least one S. epidermidis strain and/or the at least one S. hominis strain is not an antibiotic-resistant strain. S. epidermidis strains and S. hominis strains are known which have developed resistance to conventional antibiotics such as penicillin and therefore cannot be treated with corresponding preparations. It has been described in the literature that S. epidermidis strains and S. hominis strains in the natural skin flora are actively involved in controlling the growth of pathogenic organisms such as S. aureus strains. For this reason, the establishment of non-pathogenic S. epidermidis strains leads to the improvement of skin health.

The species C. acnes denotes gram-positive, anaerobic, rod-shaped bacteria, which until 2016 were taxonomically summarized as Propionibacterium acnes. The species includes pathogenic and non-pathogenic members. Pathogenic members of this species C. acnes are currently known to contribute significantly to the development of acne in humans, while non-pathogenic strains are important for maintaining a stable microbiome composition. The species can be divided into three phylogenetically distinct types, referred to herein as types I, II, and III. Type I bacteria are further divided into subtypes IA, IB, and IC. Subtype 1A is further divided into subgroups 1A1 and 1A2. Many non-pathogenic strains of C. acnes belong to (a) type I, subtype IA, subgroup IA2, (b) type I, subtype IB, or (c) type II.

It is therefore preferred that the mask composition according to the invention comprises at least one strain of the species C. acnes, wherein said strain preferably belongs to (a) type I, subtype IA, subgroup IA2, (b) type I, subtype IB, or (c) type II. In a particular embodiment, the mask composition comprises one or more C. acnes strains of type I, subtype IA, subgroup IA2. In another particular embodiment, the mask composition comprises one or more C. acnes strains of type I, subtype IB. In yet another particular embodiment, the composition comprises one or more C. acnes strains of type II.

In another particularly preferred embodiment, the mask composition according to the invention comprises in each case at least one strain of type I, subtype IA, subgroup IA2, at least one strain of type I, subtype IB, and at least one strain of type II. It is particularly preferred that the mask composition according to the invention comprises several strains of type I, subtype IA, subgroup IA2, in combination with several strains of type I, subtype IB as well as several strains of type II.

It is preferred that the mask composition of the present invention comprises a total amount of probiotically active bacteria in the range of 10⁴-10¹¹ CFU/ml, and more preferably in the range of 10⁷-10¹⁰ CFU/ml.

Preferably, the at least one probiotically active bacterial strain may be added to the mask composition of the present invention in the form of a lyophilisate. It is particularly preferred to add the at least one probiotically active bacterial strain as a lyophilisate to the oil of the mask composition. This ensures that the bacterial strain is activated only when water is added, i.e. shortly before the mask composition is used on the skin.

The mask composition of the present invention may further comprise at least one protector strain of the genus Lactobacillus that promotes colonization of the probiotically active bacterial strain. Bacteria of the genus Lactobacillus are gram-positive, microaerophilic or anaerobic bacteria taxonomically belonging to the family Lactobacillaceae. They are gram-positive, usually rod-shaped bacteria that ferment sugars and carbohydrates to lactic acids. Bacteria of the genus Lactobacillus are well known, particularly in the food industry, where they are frequently used as starter cultures in the manufacture of dairy products. The genus comprises various species which can be used without restriction in the context of the present invention.

Suitable species that may serve as protector strains in the mask composition of the present invention include Lactobacillus delbrueckii, Lactobacillus delbrueckii subsp. bulgaricus, Lactobacillus delbrueckii subsp. delbrueckii, Lactobacillus delbrueckii subsp. lactis, Lactobacillus acidophilus, Lactobacillus amylolyticus, Lactobacillus amylovorus, Lactobacillus aviaries, Lactobacillus bifermentans, Lactobacillus brevis, Lactobacillus casei, Lactobacillus coryniformis, Lactobacillus crispatus, Lactobacillus crustorum, Lactobacillus curvatus, Lactobacillus dextrinicus, Lactobacillus fermentum, Lactobacillus gasseri, Lactobacillus helveticus, Lactobacillus hilgardii, Lactobacillus iners, Lactobacillus jensenii, Lactobacillus mali, Lactobacillus oryzae, Lactobacillus panis, Lactobacillus parabuchneri, Lactobacillus paracasei, Lactobacillus paralimentarius, Lactobacillus plantarum, Lactobacillus reuteri, Lactobacillus rhamnosus, Lactobacillus ruminis, Lactobacillus salivarius, Lactobacillus selangorensis.

In another preferred embodiment, the mask composition of the present invention comprises two or more protector strains, such as 2, 3, 4, 5, 6, 7, 8, 9 or 10 Lactobacillus strains. In a particularly preferred embodiment, the at least one protector strain added to the mask composition according to the present invention is a strain of the species Lactobacillus plantarum or Lactobacillus rhamnosus. In a particularly preferred embodiment, the composition according to the invention comprises at least one strain of the species Lactobacillus plantarum and one strain of the species Lactobacillus rhamnosus. In another preferred embodiment, the mask composition according to the invention comprises several strains of the species Lacto-bacillus plantarum as well as several strains of the species Lactobacillus rhamnosus.

According to the invention, it is preferred that the at least one protector strain produces an antibiotically active substance. The antibiotically active substance may be a bacteriocin produced by the Lactobacillus and secreted into the surrounding medium. Preferably, the protector strain produces a bacteriocin selected from the group nisin, mersacidin, lactocin, acidocin B, and entereocin P. In another preferred embodiment, the protector strain contained in the mask composition of the invention produces 2 or more of said bacteriocins.

It is preferred that the mask composition of the present invention comprises a total amount of bacteria of the genus Lactobacillus in the range of 10⁴-10⁹ CFU/ml, and more preferably in the range of 10⁸-10⁸ CFU/ml. This means that the protector strains may be present in the mask composition in much lower amounts compared to the probiotically active strains. Since the protector strains only need to provide a growth advantage to the probiotically active strains during the early phase of colonization of the skin, their permanent colonization is not required. Accordingly, the protector strains can be applied to the skin at significantly lower inoculation densities.

In another advantageous embodiment, the mask composition of the present invention comprises, in addition to the at least one probiotically active bacterial strain, the flavonoid licochalcone A or derivatives thereof. Licochalcone A has the following structure:

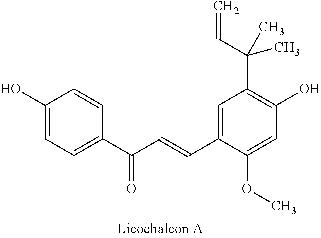 [SMILES: C=CC(C)(C)c1cc(/C=C/C(=O)c2ccc(O)cc2)c(OC)cc1O]

Licochalcone A is a chemical compound that can be extracted from the root of Chinese licorice (Glycyrrhiza inflata). It exhibits anti-inflammatory, anti-bacterial, anti-parasitic and anti-cancer effects and is regularly used in cosmetic compositions to prevent skin irritations such as redness. Licochalcone A has been shown to enhance the establishment of probiotically active strains, such as strains of S. epidermidis, S. hominis or C. acnes, on the skin.

Licochalcone A is preferably used in an amount from 0.0001 to 10 wt %, more preferably from 0.001 to 7.5 wt %, and even more preferably from 0.01 to 5 wt %, from 0.1 to 0.5 wt %, based on the total weight of the composition.

### COMPARATIVE EXPERIMENTS AND EXAMPLES

The following examples are intended to illustrate the present invention without limiting it. All indications of amounts, proportions and percentages are, unless otherwise indicated, related to the weight and the total amount or the total weight of the preparations, respectively.

The following compositions were provided, wherein only examples 2, 4 and 5 show compositions according to the invention. Examples 1, 3 and 6 are comparative examples which are not according to the invention.

| Ingredients | 1 | 2 | 3 | 4 | 5 | 6 |
|---|---|---|---|---|---|---|
| Sodium Alginate | 15.52 | 15 | 15 | 15 | 15 | 15 |
| Calcium Sulfate | 12.41 | 12 | 12 | 12 | 12 | 12 |
| Dihydrate | | | | | | |
| Tetrasodium | 2.93 | 2.83 | 2.83 | 2.83 | 2.83 | 2.83 |
| Pyrophosphate | | | | | | |
| Dicaprylyl | 17.24 | 16.66 | 16.66 | 16.66 | 16.66 | 16.66 |
| Carbonate | | | | | | |
| Glycerin | 37.59 | 36.34 | 36.34 | 36.34 | 36.34 | 36.34 |
| Polyglyceryl-4 | | 3.33 | | | | |
| Isostearate | | | | | | |
| Sodium Stearoyl | | | | 3.33 | | |
| Glutamate | | | | | | |
| Polyglyceryl-10 | | | | 3.33 | | |
| Caprylate/Caprate | | | | | | |
| PEG-7 Glyceryl | | | | | 3.33 | |
| Cocoate | | | | | | |
| Potassium Cetyl | | | | | | 2 |
| Phosphate + | | | | | | |
| Hydrogenated | | | | | | 1.33 |
| Coco Glycerides | | | | | | |
| Silica | 10.86 | 10.5 | 10.5 | 10.5 | 10.5 | 10.5 |
| Timiron Synwhite | 3.45 | 3.34 | 3.34 | 3.34 | 3.34 | 3.34 |
| 40 (synthetic | | | | | | |
| fluorophlogopite + | | | | | | |
| CI 77891 + tin | | | | | | |
| Oxide)¹ | | | | | | |

¹Timiron ® Synwhite 40 available from EMD Performance Materials (Merck KGaA) comprising:

| | Weight | | |
|---|---|---|---|
| Chemical | proportion | Color | |
| composition | in % | Index | CAS No. |
| Fluorophlogopite | 68-76 | | 12003-38-2 |
| Titanium Dioxide | 24-31 | 77891 | 13463-67-7 |
| Tin Oxide | ≤1 | | 18282-10-5 |

After preparation, compositions 1 to 6 were mixed separately with water. The mixing ratio is given below:

| Composition | 1 | 2 | 3 | 4 | 5 | 6 |
|---|---|---|---|---|---|---|
| Weight proportion | 29 | 30 | 30 | 30 | 30 | 30 |
| of the composition | | | | | | |
| Weight proportion | 71 | 70 | 70 | 70 | 70 | 70 |
| water | | | | | | |

A visual analysis of the mixtures obtained revealed the following: The examples which are not in accordance with the invention and the mixtures containing the composition which are not in accordance with the invention (1, 3, 6) showed immediate phase separation. Consequently, these mixtures were unstable. Mixtures of the compositions according to the invention (2, 4, 5) with water were significantly more stable.

Other compositions according to the invention are listed below:

| Ingredients | 7 | 8 | 9 | 10 | 11 | 12 |
|---|---|---|---|---|---|---|
| Sodium Alginate | 15 | 15 | 15 | 15 | 15 | 15.51 |
| Calcium Sulfate | 12 | 12 | 12 | 12 | 12 | 12.41 |
| Dihydrate | | | | | | |
| Tetrasodium | 2.84 | 2.5 | 3.33 | 2.84 | 2.84 | 2.93 |
| Pyrophosphate | | | | | | |
| Dicaprylyl | 16.67 | 16.67 | 16.67 | 23.33 | 0 | 0 |
| Carbonate | | | | | | |
| Dimethicone | 0 | 0 | 0 | 0 | 16.67 | 0 |
| Caprylic/Capric | 0 | 0 | 0 | 0 | 0 | 17.25 |
| Triglyceride | | | | | | |
| Jojoba Oil | 0 | 0 | 0 | 0 | 0 | 0 |
| Glycerin | 36.33 | 36.67 | 35.84 | 29.67 | 36.33 | 37.59 |
| Polyglyceryl-4 | 0 | 3.33 | 0 | 3.33 | 0 | 3.44 |
| Isostearate | | | | | | |
| Polyglyceryl-10 | 0 | 0 | 3.33 | 0 | 3.33 | 0 |
| Caprylate/Caprate | | | | | | |
| TEGO ® Care | 3.33 | 0 | 0 | 0 | 0 | 0 |
| LTP²(Sorbitan | | | | | | |
| Laurate (and) | | | | | | |
| Polyglyceryl-4 | | | | | | |
| Laurate (and) | | | | | | |
| Dilauryl Citrate) | | | | | | |
| Silica | 10.5 | 10.5 | 10.5 | 10.5 | 10.5 | 10.87 |
| Tim iron | 3.33 | 3.33 | 3.33 | 3.33 | 3.33 | 0 |
| Synwhite 40 | | | | | | |

²From Evonik

| Ingredients | 13 | 14 | 15 | 16 | 17 | 18 |
|---|---|---|---|---|---|---|
| Sodium Alginate | 15.51 | 15 | 15 | 15 | 15 | 15 |
| Calcium Sulfate | 12.41 | 12 | 12 | 12 | 12 | 12 |
| Dihydrate | | | | | | |
| Tetrasodium | 2.93 | 2.83 | 2.83 | 2.83 | 2.83 | 2.83 |
| Pyrophosphate | | | | | | |
| Dicaprylyl | 0 | 0 | 0 | 0 | 16.67 | 16.67 |
| Carbonate | | | | | | |
| Dimethicone | 0 | 16.67 | 0 | 0 | 0 | 0 |
| Caprylic/Capric | 0 | 0 | 16.66 | 0 | 0 | 0 |
| Triglyceride | | | | | | |
| Jojoba Oil | 17.25 | 0 | 0 | 16.67 | 0 | 0 |
| Glycerin | 37.59 | 0 | 0 | 0 | 36.33 | 36.33 |
| Propylene Glycol | 0 | 36.33 | 0 | 0 | 0 | 0 |
| Butylene Glycol | 0 | 0 | 36.34 | 0 | 0 | 0 |
| Sorbitol | 0 | 0 | 0 | 36.33 | 0 | 0 |
| Polyglyceryl-4 | 0 | 0 | 3.34 | 0 | 0 | 0 |
| Isostearate | | | | | | |
| Polyglyceryl-10 | 3.45 | 3.34 | 0 | 3.34 | 0 | 0 |
| Caprylate/Caprate | | | | | | |
| TEGO ® Care | 0 | 0 | 0 | 0 | 3.34 | 3.34 |
| LTP²(Sorbitan | | | | | | |
| Laurate (and) | | | | | | |
| Polyglyceryl-4 | | | | | | |
| Laurate (and) | | | | | | |
| Silica | 10.86 | 10.5 | 10.5 | 10.5 | 0 | 13.83 |
| Diatomaceous | 0 | 0 | 0 | 0 | 13.83 | 0 |
| Earth | | | | | | |
| Trimiron | 0 | 3.33 | 3.33 | 3.33 | 0 | 0 |
| Synwhite 40 | | | | | | |

²From Evonik

| Ingredients | 19 | 20 | 21 | 22 | 23 | 24 |
|---|---|---|---|---|---|---|
| Sodium Alginate | 15 | 15 | 15 | 15 | 15 | 15.51 |
| Calcium Sulfate | 12 | 12 | 12 | 12 | 12 | 12.41 |
| Dihydrate | | | | | | |
| Tetrasodium | 2.84 | 2.5 | 3.33 | 2.84 | 2.84 | 2.93 |
| Pyrophosphate | | | | | | |
| Dicaprylyl | 16.67 | 16.67 | 16.67 | 23.33 | 0 | 0 |
| Carbonate | | | | | | |
| Dimethicone | 0 | 0 | 0 | 0 | 16.67 | 0 |
| Caprylic/Capric | 0 | 0 | 0 | 0 | 0 | 17.25 |
| Triglyceride | | | | | | |
| Jojoba Oil | 0 | 0 | 0 | 0 | 0 | 0 |
| Glycerin | 36.33 | 36.67 | 35.84 | 29.67 | 36.33 | 37.59 |
| Polyglyceryl-10 | 0 | 3.33 | 0 | 3.33 | 0 | 3.44 |
| stearate | | | | | | |
| Polyglyceryl-3 | 3.33 | 0 | 3.33 | 0 | 3.33 | 0 |
| methyl-glucose | | | | | | |
| distearate | | | | | | |
| Silica | 10.5 | 10.5 | 10.5 | 10.5 | 10.5 | 10.87 |
| Timiron | 3.33 | 3.33 | 3.33 | 3.33 | 3.33 | 0 |
| Synwhite 40 | | | | | | |

| Ingredients | 25 | 26 | 27 |
|---|---|---|---|
| Sodium Alginate | 15.51 | 15 | 15 |
| Calcium Sulfate | 12.41 | 12 | 12 |
| Dihydrate | | | |
| Tetrasodium | 2.93 | 2.83 | 2.83 |
| Pyrophosphate | | | |
| Dicaprylyl | 0 | 0 | 0 |
| Carbonate | | | |
| Dimethicone | 0 | 16.67 | 0 |
| Caprylic/Capric | 0 | 0 | 16.66 |
| Triglycerides | | | |
| Jojoba Oil | 17.25 | 0 | 0 |
| Glycerin | 37.59 | 0 | 0 |
| Propylene Glycol | 0 | 36.33 | 0 |
| Butylene Glycol | 0 | 0 | 36.34 |
| Sorbitol | 0 | 0 | 0 |
| Sorbitan Laurate | 0 | 1.15 | 0.21 |
| Polyglyceryl-4 | 3.45 | 2.05 | 2.84 |
| Laurate | | | |
| Dilauryl Citrate | | 0.14 | 0.29 |
| Silica | 10.86 | 10.5 | 10.5 |
| Diatomaceous | 0 | 0 | 0 |
| Earth | | | |
| Trimiron Synwhite | 0 | 3.33 | 3.33 |
| 40 | | | |

After preparation, compositions 7 to 27 were mixed in the ratio of 24:76, 27:73, 31:69 and 36:64 (composition:water), respectively.

1.-18. (canceled)

19. A cosmetic composition, wherein the composition is anhydrous and comprises
a) at least one water-soluble alginate,
b) at least one calcium salt,
c) at least one oil,
d) at least one emulsifier from the group polyglyceryl-4 isostearate, polyglyceryl-10 caprylate/caprate, polyglyceryl-10 stearate, PEG-7 glyceryl cocoate, polyglyceryl-3 methyl glucose distearate, and polyglyceryl-4 laurate.

20. The composition of claim 19, wherein the composition further comprises at least one polyol.

21. The composition of claim 20, wherein the polyol comprises one or more of propylene glycol, butylene glycol, glycerin and sorbitol.

22. The composition of claim 19, wherein the polyol comprises at least glycerin.

23. The composition of claim 20, wherein a total concentration of the one or more polyols is from 15 wt % to 50 wt %, based on a total weight of the composition.

24. The composition of claim 19, wherein the at least one water-soluble alginate comprises alginic acid and/or a salt of alginic acid.

25. The composition of claim 19, wherein the at least one water-soluble alginate comprises sodium alginate.

26. The composition of claim 19, wherein a total concentration of the at least one water-soluble alginate is from 5 wt % to 30 wt %, based on a total weight of the composition.

27. The composition of claim 19, wherein a total concentration of the at least one calcium salt is from 5 wt % to 20 wt %, based on a total weight of the composition.

28. The composition of claim 19, wherein the at least one calcium salt comprises one or both of calcium sulfate and calcium sulfate dihydrate.

29. The composition of claim 19, wherein a total concentration of the at least one oil is from 2 wt % to 40 wt %, based on a total weight of the composition.

30. The composition of claim 19, wherein the at least one oil comprises at least one of ethyl hexyl stearate, octyl cocoate, coco glycerides, dicaprylyl carbonate, caprylic capric triglyceride, cetearyl isononanoate, C12-15 alkyl benzoate, and isopropyl palmitate, a total concentration of these oils being from 5 wt % to 25 wt %, based on a total weight of the composition.

31. The composition of claim 19, wherein the at least one emulsifier comprises at least one of polyglyceryl-4 isostearate, polyglyceryl-10 caprylates/caprates, polyglyceryl-10 stearate, PEG-7 glyceryl cocoate, polyglyceryl-3 methyl glucose distearate, and polyglyceryl-4 laurate, a total concentration of these emulsifiers being from 0.5 wt % to 11 wt %, based on a total weight of the composition.

32. The composition of claim 19, wherein the composition further comprises one or more complexing agents for calcium ions.

33. The composition of claim 32, wherein a concentration of the one or more complexing agents is from 0.2 wt % to 5 wt %, based on a total weight of the composition.

34. The composition of claim 19, wherein the composition further comprises at least one probiotically active bacterial strain which, when colonized on skin, has a positive effect on skin health.

35. The composition of claim 34, wherein the composition further comprises at least one protector strain of genus Lactobacillus which promotes a colonization of the probiotically active bacterial strain.

36. The composition of claim 19, wherein the composition further comprises licochalcone A.

37. A method of preparing a cosmetic preparation for use as a mask which can be peeled off after application to skin, wherein the method comprises
i. providing the anhydrous cosmetic composition of claim 19,
ii. mixing the composition with water to provide an emulsion for application to the skin.

38. The method of claim 37, wherein the composition of claim 19 is mixed with water in a weight ratio to water of from 10:90 to 60:40.
